(19) United States
(12) Patent Application Publication
(10) Pub. No.: US 2024/0033058 A1
(43) Pub. Date: Feb. 1, 2024

(54) ORDER ASSIGNMENT METHOD FOR DENTAL RESTORATIONS

(71) Applicant: Ivoclar Vivadent AG, Schaan (LI)

(72) Inventors: Theresa Sujata Maria Senti, Triesenberg (LI); Sebastian Gassner, Triesenberg (LI); Mathias Burtscher, Feldkirch (AT)

(21) Appl. No.: 18/359,073

(22) Filed: Jul. 26, 2023

(30) Foreign Application Priority Data
Jul. 27, 2022 (EP) 22187307.8

(51) Int. Cl. A61C 13/00; G16H 30/20

(52) U.S. Cl. CPC A61C 13/00; G16H 30/20

(57) ABSTRACT

A method for assigning an order identification to a manufactured dental restoration, including the steps of scanning (S101) the manufactured restoration to generate an actual data set representing the actual shape of the manufactured restoration; retrieving (S102) a data set representing a nominal shape of a digital restoration and to which an order identification is assigned; comparing (S103) the actual shape with the nominal shape; and detecting (S104) the order identification of the data set when the actual shape corresponds to the nominal shape.

### CROSS-REFERENCE TO RELATED APPLICATIONS

This application claims priority to European Application No. 22187307.8 filed on Jul. 27, 2022, the disclosure of which is incorporated herein by reference in its entirety.

### TECHNICAL FIELD

The present invention relates to a method for assigning an order identification to a manufactured dental restoration, an assignment device for assigning an order identification to a manufactured dental restoration, and a computer program for assigning an order identification to a manufactured dental restoration.

### BACKGROUND

If several dental restorations are manufactured in one manufacturing process, the problem often arises that they have to be assigned to a manufacturing order. An optically manual assignment of the manufactured restorations by a user is very error-prone.

During an inspection of outgoing goods, a comparison is made, for instance, with a model produced especially for this purpose. In addition to the fit, the color of the dental restoration is also checked. This is done using a shade guide based on a four-eye principle by two different users. These approaches are error-prone and time-consuming. In some cases, the model is produced specifically for the above activities. This in turn means additional time and material expenditure.

In the production of dental restorations, there are also batch processes in which several dental restorations are produced in a single operation, such as the milling of several restorations from a blank or the sintering of restorations. After completion of the batch process, it is difficult to reassign the individual restorations to the correct order. This presents an enormous challenge for small objects that look similar, such as single-tooth crowns in the posterior region, veneers or inlays.

US 20190130596, 20210007833, 20140234803, 20190295254, 2018035266, and 6,968,247, are directed to systems and methods related to dental materials and dental structures and are hereby incorporated by reference in their entirety.

### SUMMARY OF THE INVENTION

It is therefore the technical object of the present invention to enable fast and reliable order assignment of dental restorations.

This technical object is solved by subject-matter according to the independent claims. Technically advantageous embodiments are the subject-matter of the dependent claims, the description and the drawings.

According to a first aspect, the technical object is solved by a method for assigning an order identification to a manufactured dental restoration, comprising the steps of scanning the manufactured restoration to generate an actual data set representing the actual shape of the manufactured restoration; retrieving a data set representing a nominal shape of a digital restoration and to which an order identification is assigned; comparing the actual shape with the nominal shape; and detecting the order identification of the data set when the actual shape corresponds to the nominal shape. Scanning can obtain an actual data set that represents the two-dimensional or three-dimensional shape of the manufactured restoration. The retrieved data set can also represent the two-dimensional or three-dimensional shape of a restoration. This achieves the technical advantage, for example, that the manufactured dental restoration can be reliably recognized and automatically processed further on the basis of the order identification. The process enables the unambiguous assignment of a dental restoration to an order. It is also possible to compare the nominal properties of the restoration, such as shape, color or size, with the actual properties of the manufactured restoration.

In a technically advantageous embodiment of the method, a further data set is retrieved which represents a nominal shape of a digital restoration and to which an order identification is assigned if the actual shape deviates from the nominal shape. This achieves the technical advantage, for example, that the comparison can be carried out until an order identification has been found.

In another technically advantageous embodiment of the method, a further data set is retrieved, which represents a nominal shape of a digital restoration and to which an order identification is assigned, until the actual shape corresponds to the nominal shape. Several data sets can be retrieved from a list, which are checked one after the other until the actual shape corresponds to the nominal shape. After a predetermined number of steps, the retrieval of further data sets can be aborted to avoid an endless loop. This also achieves the technical advantage, for example, that the comparison can be carried out until an order identification has been found.

In another technically advantageous embodiment of the method, the data set which represents a nominal shape of a digital restoration and to which an order identification is assigned is retrieved from a database. This achieves the technical advantage, for example, that the data sets for a plurality of restorations can be retrieved individually in a fast manner.

In another technically advantageous embodiment of the method, the scanning process obtains an actual data set that represents a spatial actual shape of the manufactured restoration. The spatial actual shape is a three-dimensional shape of the manufactured restoration. This achieves the technical advantage, for example, that the accuracy of the method is improved.

In another technically advantageous embodiment of the method, the manufactured restoration is placed in a tray with the order identification. This achieves the technical advantage, for example, of simplifying further processing and handling of the manufactured dental restoration.

In another technically advantageous embodiment of the method, the manufactured restoration is labeled with the order identification. This also achieves the technical advantage, for example, of simplifying further processing and handling of the manufactured dental restoration.

In another technically advantageous embodiment of the method, several manufactured dental restorations are scanned simultaneously and an actual data set is generated for each of the scanned dental restorations, which represents the actual shape of the respective manufactured restoration. This also achieves the technical advantage, for example, that the speed of the method is improved. In addition, the work of the user is also made easier, as manual sorting and separation of the dental restorations is no longer necessary.

In a further technically advantageous embodiment of the method, each of the actual shapes is compared with the nominal shape. This also achieves the technical advantage, for example, that the method can be accelerated.

In another technically advantageous embodiment of the method, the restoration whose actual shape corresponds to the nominal shape is optically highlighted from the plurality of dental restorations. The optical highlighting can be performed, for example, by a light source which illuminates the restoration in a selective or planar manner relative to the other restorations. For example, the dental restoration whose actual shape corresponds to the nominal shape is highlighted in a light cone. This also achieves the technical advantage, for example, that the corresponding restoration can be identified from a plurality of restorations. The user can receive feedback as to which manufactured restoration belongs to which order.

According to a second aspect, the technical object is solved by an assignment device for assigning an order identification to a manufactured dental restoration, comprising a scanner for scanning the manufactured restoration to generate an actual data set representing the actual shape of the manufactured restoration; a retrieval device for retrieving a data set representing a nominal shape of a digital restoration and to which an order identification is assigned; a comparison device for comparing the actual shape with the nominal shape; and a detection device for detecting the order identification of the data set when the actual shape corresponds to the nominal shape. Scanning can obtain an actual data set that represents the two-dimensional or three-dimensional shape of the manufactured restoration. The retrieved data set can also represent the two-dimensional or three-dimensional shape of a restoration. The assignment device achieves the same technical advantages as the method according to the first aspect.

In a technically advantageous embodiment of the assignment device, the scanner is configured to generate an actual data set that represents a spatial actual shape of the manufactured restoration. The spatial actual shape is a three-dimensional shape of the manufactured restoration. This also achieves the technical advantage, for example, that the accuracy of assignment can be improved.

In another technically advantageous embodiment of the assignment device, the assignment device comprises a labeling device for labeling the manufactured restoration with the order identification or an insertion device for inserting the manufactured restoration into a tray with the order identification. This also achieves the technical advantage, for example, that further processing and handling of the dental restoration is simplified.

In another technically advantageous embodiment of the assignment device, the assignment device comprises a highlighting device for optically highlighting that dental restoration from several dental restorations whose actual shape corresponds to the nominal shape. This also achieves the technical advantage, for example, that the matching dental restoration can be identified in a simple manner.

According to a third aspect, the technical object is solved by a computer program, comprising instructions which, when the computer program is executed by an assignment device, cause the assignment device to execute the method according to the first aspect. The computer program may be executed, for example, on a computer with a camera, a tablet PC, or a cell phone. These devices may also be embedded in an assignment device comprising further features. The computer program is stored in a memory of the computer and executed by a processor. In the process, the data sets are processed. The computer program achieves the same technical advantages as the method according to the first aspect.

According to this third aspect, a computer program product comprises computer program code which is stored on a non-transitory machine-readable medium and comprises computer instructions executable by a processor to perform the method described above.

### BRIEF DESCRIPTION OF THE DRAWINGS

Exemplary embodiments of the invention are shown in the drawings and are described in more detail below, in which:

FIG. 1 shows a schematic view of an assignment device for assigning an order identification;

FIG. 2 shows a schematic view of a highlighting device for highlighting a dental restoration; and

FIG. 3 shows a block diagram of a method for assigning an order identification.

### DETAILED DESCRIPTION

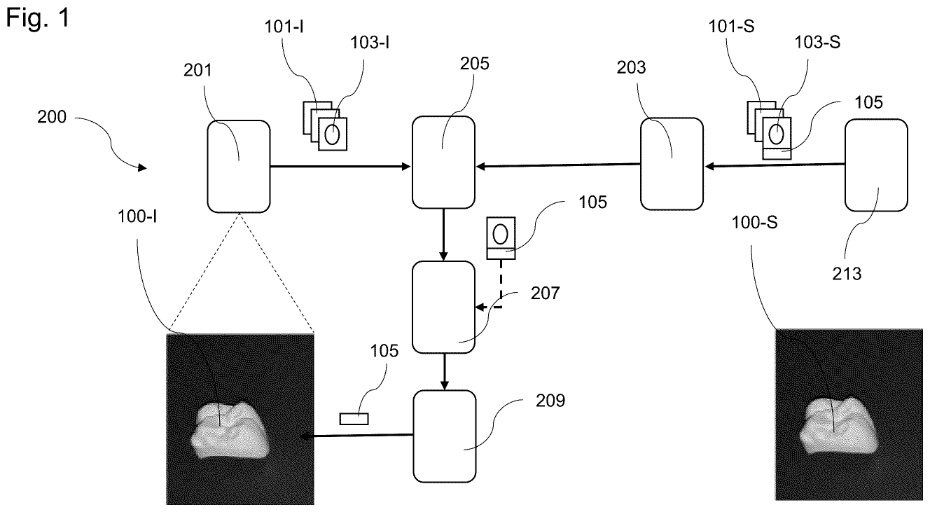

FIG. 1 shows a schematic view of an assignment device 200 for assigning an order identification to a manufactured dental restoration 100-I. The assignment device 200 comprises a scanner 201 for scanning the manufactured restoration 100-I to generate an actual data set 101-I representing the actual shape 103-I of the manufactured restoration 100-I. The scanner 201 can detect manufactured dental restorations 100-I in a workspace using camera-based object recognition so that they can be classified subsequently.

The dental restoration 100-I is, for example, a milled, pre-dried restoration manufactured from a zirconium oxide ceramic. The dental restoration 100-I may consist of a fully anatomical crown to a fully anatomical 4-unit bridge. For example, the dental restoration 100-I may also be another dental object, such as a crown, bridge, veneer, abutment, inlay, onlay, splint, or partial or full prosthesis. In general, the dental restoration 100-I can be any dental object that is to be custom fabricated as part of a dental treatment.

The scanner 201 may use an imaging process and is formed, for example, by an electronic camera with CCD array that captures a two-dimensional image of the dental restoration 100-I, such as a bitmap file. From this two-dimensional image, the two-dimensional actual shape 103-I of the manufactured restoration 100-I can be determined. This can be compared with renderings of different perspectives of the digital simulation until a match is found. Using several different images and constant parameter settings, the number of restorations 100-I can be calculated, the largest restoration 100-I can be determined and the position of the restoration 100-I can be checked. The number of restorations 100-I found in a scene can be narrowed down by a minimum evaluation. This evaluation and other parameter settings can then remain the same for several images.

However, the scanner 201 may also comprise a stereoscopic camera, a structured-light camera, and/or a time-of-flight (ToF) scanner that can capture the dental restoration 100-I in three dimensions. Such a scanner 201 can generate an actual data set 101-I that represents the three-dimensional actual shape 103-I of the manufactured restoration 100-I. The dental restorations 100-I can be captured individually, or a larger area in which several restorations are arranged can be captured simultaneously.

The stereoscopic camera is a special device for taking stereoscopic photographs. From these stereoscopic photographs, the three-dimensional shape of the dental restoration 100-I can be calculated.

The structured-light camera uses an active triangulation method, namely structured-light projection, to image the restoration. Surface-based 3D matching can be used to detect the dental restorations 100-I. This is particularly suitable for searching for different dental restorations 100-I, any number of dental restorations 100-I and dental restorations 100-I in any position. Structured-light projection is technically particularly suitable due to high speed during recording, great robustness to ambient light and high accuracy for objects with different geometries and opaque materials.

The ToF scanner is a 3D camera system that measures distances using the time-of-flight method. For this purpose, the dental restoration 100-I is illuminated by means of a light pulse, and the camera measures the time required for the light to reach the dental restoration 100-I and back again for each image point. The time required is directly proportional to the distance. The camera thus provides the distance of the dental restoration 100-I imaged on it for each image point. The principle corresponds to laser scanning with the advantage that an entire scene is recorded at once and does not have to be scanned.

Further, the assignment device 200 comprises a retrieval device 203 for retrieving a data set 101-S that represents a nominal shape 103-S of a digital restoration 100-S and to which an order identification 105 is assigned. The data set 101-S may comprise CAD data of the digital restoration 100-S, such as a CAD model.

The order identification 105 is used to identify an order on the basis of which the manufactured dental restoration 100-I has been manufactured. The order identification 105 comprises, for example, an order number or technical data for the manufacture of the dental restoration 100-I. The retrieval device 203 is in communication, for example via an interface, with a database 213 from which the data sets 101-S of the digital restoration 100-S can be retrieved. Retrieval of the data sets 101-S may be performed individually or based on a predetermined list of nominal data sets 101-S.

Further, the assignment device 200 comprises a comparison device 205 for comparing the actual shape 103-I with the nominal shape 103-S. The assignment device 200 is formed by software, for example, and is capable of calculating a deviation between the actual shape 103-I and the nominal shape 103-S. If a deviation found between the actual shape 103-I and the nominal shape 103-S falls below a predetermined value, the actual shape 103-I of the manufactured dental restoration 100-I is assigned to the nominal shape 103-S of the digital restoration 100-S. However, the comparison device 205 can also select the nominal data set 103-S by determining, from a plurality of nominal shapes 103-S, the one that has the smallest deviation or greatest similarity to the actual shape 103-I.

This can be used, for example, to compare restorations 100-I manufactured in a laboratory with digital restorations 100-S stored as an STL file in a database 213 or to search for a CAD model in the image.

The software can establish a connection between the orders present in the system and the dental restorations 100-I manufactured. This can be done, for example, using the CAD data of the restoration 100-S, which is compared to the recorded dental restoration 100-I. The results of the comparison may be output to a display unit, such as a cell phone, a tablet, a computer screen, or a display on the device.

Further, the assignment device 200 comprises a detection device 207 for detecting the order identification 105 of the data set 101-S, when the actual shape 103-I corresponds to the nominal shape 103-S. The detection device 207 may also be formed by the software extracting the order identification 105 from the found data set 101-S. The order identification 105 is then assigned to the scanned restoration 100-I. The software may be executed on embedded hardware that provides the desired requirements.

Once the dental restoration 100-I is identified, a labeling device 209 can be used to label the manufactured restoration 100-I with the order identification 105. For example, the labeling device 209 may engrave an engraving on the inside of the dental restoration 100-I or apply a label to the dental restoration 100-I. The engraving with the order identification 105 may be created using a suitable laser, for example. The label may be generated using a label printer.

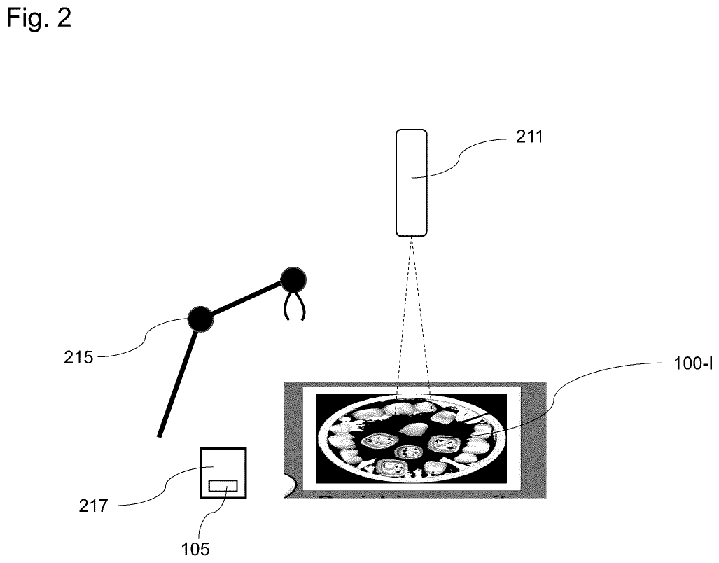

FIG. 2 shows a schematic view of a highlighting device 211 for highlighting a manufactured dental restoration 100-I associated with a determined order identification 105. In the event of a successful order assignment, the searched restoration 100-I can be visually highlighted by the highlighting device 211.

For example, the dental restoration 100-I may be arranged on a display device or other surface that can be selectively activated to give light, such as an LED field. When an assigned order is selected, the area under the searched dental restoration 100-I is automatically activated to give light, while the other areas remain in darkness.

However, the highlighting device 211 can also focus light on the searched dental restoration 100-I by means of an optical system. In this way, the dental restoration 100-I appears in a cone of light. The user can thus receive feedback in a simple and intuitive manner as to which manufactured restoration 100-I belongs to which order identification 105. This is particularly advantageous if several dental restorations 100-I are produced in one work step and leave the system at the same time.

The detected dental restorations 100-I may be visually displayed on a display unit, where they may also be provided with the order identification 105. Conversely, by selecting the dental restoration 100-I on a displayed image, the associated order identification 105 may be displayed.

In addition, a single restoration 100-I can be manually presented to the assignment device 200 for scanning. This can be done by holding it in front of the scanner 201. The associated order identification 105 is then automatically displayed.

However, the assignment device 200 may also comprise a controlled gripper arm or robot arm as an insertion device 215 that places the identified dental restoration 100-I into a tray 217 or box that is provided with the order identification 105. This may facilitate handling in the manufacture of the dental restorations 100-I.

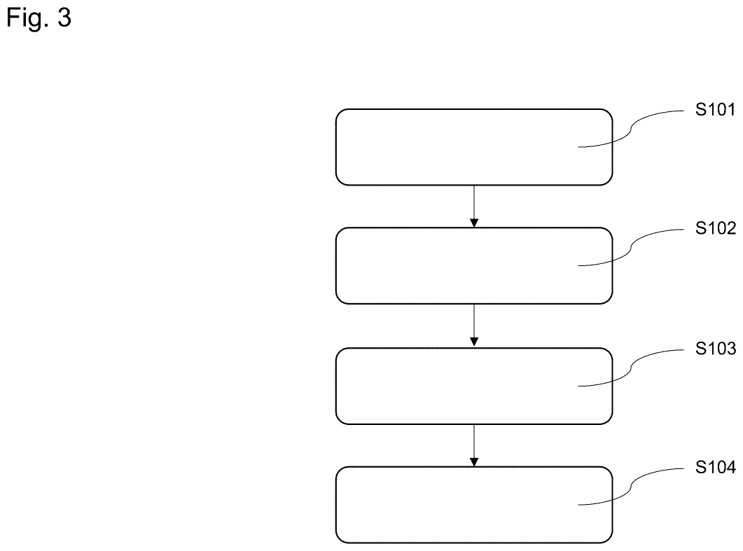

FIG. 3 shows a block diagram of a method for assigning an order identification to the manufactured dental restoration 100-I. The method comprises the step S101 of scanning the manufactured restoration 100-I to generate an actual data set 101-I representing the actual shape 103-I of the manufactured restoration 100-I. In step S102, the data set 101-S representing a nominal shape 103-S of a digital restoration 100-S is retrieved and is assigned an order identification 105. In step S103, the actual shape 103-I is compared to the nominal shape 103-S and a measure of a deviation between the two shapes 103-I and 103-S is calculated. In step S104, the order identification 105 of the data set 101-DB is detected if the actual shape 103-I corresponds to the nominal shape 101-S. For example, the actual shape 103-I corresponds to the nominal shape 101-S if the measure of deviation is below a predetermined value.

The method is used to classify the restorations 100-I and enables the unambiguous assignment of a dental restoration 100-I to an order. The method reduces assignment errors and increases efficiency. In addition, the amount of material required for the production of dental restorations is reduced, since the production of comparison models can be dispensed with. The method can be carried out, for example, on a cell phone, a tablet PC or a desktop PC with a camera.

For example, before a dental restoration 100-I leaves the dental laboratory, it is checked one last time to ensure that all requirements are met. This includes in particular the fit on the model, i.e. contact points or occlusion, but also the color of the restoration 100-I.

However, with the increase in digital incoming orders, fewer and fewer models are being produced, as they often play a subordinate role in the manufacture of the restoration 100-I. It is often only a single try-in step for which a model is produced. This expenditure of time and, above all, material can be saved.

The method allows the manufactured restoration 100-I to be scanned again before shipping and inserted in the digital model or in an intraoral scan using software. In this way, the fit of the manufactured dental restoration 100-I can also be checked (digital try-in). Scanning additionally allows the layer thickness of the manufactured restoration 100-I to be checked.

All of the features explained and shown in connection with individual embodiments of the invention may be provided in different combinations in the subject matter of the invention to simultaneously realize their beneficial effects.

All method steps can be implemented by devices which are suitable for executing the respective method step. All functions that are executed by the features in question can be a method step of a method.

The scope of protection of the present invention is given by the claims and is not limited by the features explained in the description or shown in the figures.

### LIST OF REFERENCE SIGNS

100-I manufactured restoration
100-S digital restoration
101-I actual data set
101-S nominal data set
103-I actual shape
103-S nominal shape
105 order identification
200 assignment device
201 scanner
203 retrieval device
205 comparison device
207 detection device
209 labeling device
211 highlighting device
213 database
215 insertion device
217 tray

1. A method for assigning an order identification to a manufactured dental restoration, comprising
scanning the manufactured restoration to generate an actual data set representing the actual shape of the manufactured restoration;
retrieving a data set which represents a nominal shape of a digital restoration and to which an order identification is assigned;
comparing the actual shape with the nominal shape; and
detecting the order identification of the data set, if the actual shape corresponds to the nominal shape.

2. The method according to claim 1,
wherein a further data set is retrieved which represents a nominal shape of a digital restoration and to which an order identification is assigned if the actual shape deviates from the nominal shape.

3. The method according to claim 1,
wherein a further data set is retrieved which represents a nominal shape of a digital restoration and to which an order identification is assigned until the actual shape corresponds to the nominal shape.

4. The method according to claim 1,
wherein the data set which represents a nominal shape of a digital restoration and to which an order identification is assigned is retrieved from a database.

5. The method according to claim 1,
wherein an actual data set representing a spatial actual shape of the manufactured restoration is obtained by the scanning.

6. The method according to claim 1,
wherein the manufactured restoration is placed in a tray having the order identification.

7. The method according to claim 1,
wherein the manufactured restoration is labeled with the order identification.

8. The method according to claim 1,
wherein a plurality of manufactured dental restorations are scanned simultaneously and an actual data set is generated for each of the scanned dental restorations, the actual data set representing the actual shape of the respective manufactured restoration.

9. The method according to claim 8,
wherein each of the actual shapes is compared to the nominal shape.

10. The method according to claim 9,
wherein from the plurality of dental restorations, the one whose actual shape corresponds to the nominal shape is optically highlighted.

11. An assignment device for assigning an order identification to a manufactured dental restoration, comprising:
a scanner for scanning the manufactured restoration to generate an actual data set representing the actual shape of the manufactured restoration;
a retrieval device for retrieving a data set which represents a nominal shape of a digital restoration and to which an order identification is assigned;
a comparison device for comparing the actual shape with the nominal shape; and
a detector for detecting the order identification of the data set when the actual shape corresponds to the nominal shape.

12. The assignment device according to claim 11,
wherein the scanner is configured to generate an actual data set representing a spatial actual shape of the manufactured restoration.

13. The assignment device according to claim 11,
wherein the assignment device comprises a labeling device for labeling the manufactured restoration with the order identification or an insertion device for inserting the manufactured restoration into a tray with the order identification.

14. The assignment device according to claim 11,
wherein the assignment device comprises a highlighting device for optically highlighting that dental restoration from a plurality of dental restorations whose actual shape corresponds to the nominal shape.

15. A computer program product comprising computer program code which is stored on a non-transitory machine-readable medium comprising computer instructions executable by a processor to perform the method according to claim 1.
